(19) United States
(12) Patent Application Publication
(10) Pub. No.: US 2002/0106682 A1
(43) Pub. Date: Aug. 8, 2002

(54) Method and device for detecting DNA

(75) Inventors: Jeong Gun Lee, Kyonggi-do (KR); Kyu Sik Yun, Seoul (KR); Je Kyun Park, Seoul (KR); Su Hyeon Kim, Seoul (KR); Tae Han Kim, Seoul (KR); Sang Eun Lee, Seoul (KR)

Correspondence Address: MORGAN LEWIS & BOCKIUS LLP, 1111 PENNSYLVANIA AVENUE NW, WASHINGTON, DC, 20004, US

(73) Assignee: LG Electronics Inc.

(21) Appl. No.: 10/000,091

(22) Filed: Dec. 4, 2001

(30) Foreign Application Priority Data
Feb. 3, 2001 (KR) 2001-5217

(51) Int. Cl. C12Q 1/68; C12M 1/34

(52) U.S. Cl. 435/6; 435/287.2

(57) ABSTRACT

Method and device for detecting DNA by using electrochemiluminescence, wherein a probe DNA is immobilized on a chip, a target DNA is placed on the chip having the probe DNA immobilized thereon, for hybridization of the probe DNA and the target DNA, and an intercalator is fixed to the hybridized DNA. Then, an electrochemiluminescent reaction fluid is introduced into the chip having the DNA with a fixed intercalator, a preset voltage is applied to the chip for causing reaction between the intercalator and the electrochemiluminescent reaction fluid, and a light from the reaction is detected, and analyzed, whereby making a DNA detection process fast and simple, and a system of the device for detecting a DNA simple, to permit a device price low, and the DNA detection accurate.

This application claims the benefit of the Korean Application No. PO1-5217 filed on Feb. 3, 2001, which is hereby incorporated by reference.

### BACKGROUND OF THE INVENTION

1. Field of the Invention

The present invention relates to a method for detecting DNA, and more particularly, to method and device for detecting DNA by using electrochemiluminescence.

2. Background of the Related Art

In general, nucleic acid analysis of deoxyribonucleic acid (DNA), or ribonucleic acid (RNA), or the like is employed in many fields, such as biological research, medical diagnosis, search for new drug, legal medicine, and etc.

In the southern blotting analysis, an approach for detection and identification of a DNA having a specific base sequence, DNA fragments are separated by size in the electrophoresis and moved on a solid substrate, such as a nitrocellulose, or nylon membrane so that the relative positions of the DNA fragments are maintained. Subsequently, DNA having a base sequence intended to observe labeled with a radioactive isotope is placed as a probe into the DNA fragments immobilized on the solid substrate. The DNA placed as a probe can bind to the DNA fragments that have a complementary sequence by way of hybridization, which permits detection of a position, and identification, of the DNA having the specific sequence.

This approach has been extended to the Northern blotting analysis for analyzing RNA, of which principle is different not so much from the Southern blotting. The Northern blotting is employed when it is intended to detect RNA having a specific sequence included in a sample, and obtain information on an amount and size of the RNA. A sample RNA fragmented by sizes using electrophoresis in agarose gel, or polyacyrylamide gel under a denatured state is moved, and adsorbed to a surface of a nitrocellulose membrane by capillary action, and fixed by direction of a UV ray, or heating. As a probe having a sequence complementary to the immobilized RNA, a radiolabeled, or non-radiolabeled nucleic acid fragment is employed. Non-specific probe nucleic acid not come from the complementary bonding with an immobilized RNA is removed by washing with gradually decreased salt concentration, or gradually elevated temperature, a desired RNA is detected by autoradiography, or phosphorimage analysis. Other than this, a desired nucleic acid may be analyzed by the mutation searching, or the DNA sequencing, or the like.

In general, the related art nucleic acid analyses described above require much labor, skills, much time, and much resource. In order to solve those problems, a DNA chip is developed, in which a two dimensional array of nucleic acids disposed onto solid substrate at known positions. The DNA chip permits search of genes from a few hundreds at the smallest, and to more than 400,000, quickly.

The DNA chip, having an array of high concentration DNA fragments with a great variety of base sequences on a small substrate surface, is used for obtaining information of DNA in an unknown sample by hybridization with an immobilized DNA and a complementary DNA in the unknown sample. The hybridization is formation of a double-stranded DNA caused by bonding of subsequences having complementary base sequences coming from hydrogen bonding between adenine(A)-thymine(T), and guanine(G)-cytosine(C), in DNA bases. Therefore, information on the DNA base sequence in the sample can be known by labeling the double-stranded DNA after the hybridization DNA probing with the DNA sample. In general, for detecting a result of reaction of the DNA hybridization, the radioautography has been the most widely used the most in current molecular biology, in which a target DNA is labeled with radioactive isotope. ³²P, or the like, is used as the radioactive isotope, bonding of the labeled target DNA and the probe DNA is detected on photographic films. Though this method can be applicable easily as no basic knowledge is required, this method has problems in that a result of analysis cannot be known immediately since the analysis requires a time period in a range from a few hours to one day, shows a resolution merely in an order of 0.1 μm-10 μm, and has a problem of stability of the radioactive isotope.

Accordingly, the laser-induced fluorescence (LIF) analysis is currently used the most widely, which is advantageous in that different fluorescent materials can be used, a good resolution can be obtained, and a result can be obtained, immediately. If a charge coupled device (CCD) camera is introduced into a fluorescence analysis as a combination of the fluorescence analysis and an imaging technology, molecules labeled with fluorescence material can be imaged, immediately. Even if this method is used the most widely, processes for labeling with a fluorescent material, separating, and refining before detecting DNA in a sample are complicated, and expensive equipments, such as laser, and optical testing accessories, are required. Moreover, for scanning a two dimensional substrate surface, an expensive image scanner should be required.

As another method, there is an optical analysis by using a waveguide. An evanescent wave is formed by a two dimensional optical waveguide and light scattering, and the evanescent wave is made to be scattered at a label adsorbed to a DNA capture region on a surface of the waveguide. Particles placed as labels are concentrated only at a point probe oligomers and DNA fragments bond, at which a light is scattered. Since no washing is required, making the process very convenient, and requiring no time delay in a detecting, a bonding pattern in the chip can be analyzed by means of a CCD camera, or 8 mm video, immediately.

Also, there are many researches on a method for detecting a DNA bonding without a label by means of surface plasmon resonance (SPR). In the SPR, a variation of a refractive index caused by a thickness variation of surface plasmon formed on, and bonded to, a thin metal surface is detected, eventually, making an easy detection of DNA bonding available without any label. However, such a method has a poor sensitivity. As an electrochemical method for detecting a DNA hybridization, a bonding is used, in which a metal complex having an electrochemical activity with a double-stranded DNA are bonded, which has a very simple system, to make a low priced detecting device available, but with a poor sensitivity.

Because all of current methods of DNA hybridization detection have various disadvantages, development of a new detection method is required, particularly, a method for detecting a DNA hybridization that has a good sensitivity, and quickly, and requires no step of making a covalent bonding of the sample with a label material in advance, and a small sized, and low priced system for development of a portable detecting device, are required.

### SUMMARY OF THE INVENTION

Accordingly, the present invention is directed to method and device for detecting DNA that substantially obviates one or more of the problems due to limitations and disadvantages of the related art.

An object of the present invention is to provide method and device for detecting DNA, which can detect the DNA without labeling.

Another object of the present invention is to provide method and device for detecting DNA, which can detect the DNA, quickly, and at a low cost.

Additional features and advantages of the invention will be set forth in the description which follows, and in part will be apparent from the description, or may be learned by practice of the invention. The objectives and other advantages of the invention will be realized and attained by the structure particularly pointed out in the written description and claims hereof as well as the appended drawings.

To achieve these and other advantages and in accordance with the purpose of the present invention, as embodied and broadly described, the method for detecting DNA includes the steps of (a) immobilizing a probe DNA on a chip, (b) placing a target DNA on the chip having the probe DNA immobilized thereon, for hybridization of the probe DNA and the target DNA, (c) fixing an intercalator to the hybridized DNA, (d) introducing a electrochemiluminescent reaction fluid into the chip having the DNA with a fixed intercalator, (e) applying a preset voltage to the chip for causing reaction between the intercalator and the electrochemiluminescent reaction fluid, and (f) detecting, and analyzing a light from the reaction.

The step (a) includes the steps of washing an electrode of gold on the chip of silicon, or borosilicate glass with piranha solution and water in succession for the first time, dipping the gold electrode in a mixed solution (the probe DNA and ω-hydroxy-undecanethiol 3, or 3-mercaptopropionic acid are dissolved in an ethanol/octane mixed solvent) containing the probe DNA, and washing the gold electrode with ethanol and water for the second time in order to remove unfixed probe DNA.

The probe DNA has a thiol functional group at a 5′-phosphate position, and the intercalator is one selected from daunorubicin, nogalamycin, doxorubicin, and DAPI(4′,6-diamidino-2-phenylindole), or, one selected from a material obtained by bonding proline, oxalic acid, or TPA (tripropylamine) with Hoechst 33258, quinacrine, or acridine orange.

The electrochemiluminescent reaction fluid is one selected from Tris(2.2′-bipridiyl)ruthenium(II)[Ru(bpy)₃²⁺], Tris(2.2′-bipridiyl)osmium(II)[Os(bpy)₃²⁺], Tris(1,10-phenanthroline) ruthenium(II) [Ru(phen)₃²⁺] or Tris(1,10-phenanthroline) ruthenium(II) [Ru(phen)₃²⁺]

In another aspect of the present invention, there is provided a device for detecting a DNA including a fastening part for fastening a DNA chip having a plurality of probe DNAs different from one another on an electrode, a sample supplying part for supplying samples introduced for detecting a desired DNA from the DNA chip, an injecting part for injecting the samples supplied from the sample supplying part to the DNA chip, a power source part for applying a voltage to the electrode of the DNA so that the DNA makes reaction with the sample to cause electrochemiluminescence, an optical detection part for detecting a light of the electrochemiluminescence to analyze the DNA, and a discarding part for discarding unnecessary sample from the sample supplied to the DNA chip.

The sample supplying part includes an electrochemiluminescent reaction fluid supplying part for supplying electrochemiluminescent reaction fluid, such as Tris(2.2′-bipridiyl)ruthenium(II) [Ru(bpy)₃²⁺], Tris(2.2′-bipridiyl)osmium(II)[Os(bpy)₃²⁺] or Tris(1,10-phenanthroline) ruthenium(II) [Ru(phen)₃²⁺], a buffer solution supplying part, an intercalator supplying part, and a target DNA supplying part, and the target DNA supplying part includes a heater for heating the DNA double strand for denature the DNA double strand.

The injecting part is a precision pump, or a dispenser, the power source part is potentiostat, the optical detection part is one selected from a photomultiplier tube (PMT), an avalanche photodiode, and a cooled CCD camera.

In further aspect of the present invention, there is provided a device for detecting a DNA including a working mounter for fastening, and mounting a DNA chip having probe DNAs arranged thereon on an electrode, a sample reservoir for storage sample introduced for detecting a desired DNA from the DNA chip, a first driving part for moving the working mounter to the sample reservoir for dipping the DNA chip mounted on the working mounter in the sample, a second driving part for moving the sample reservoir for dipping the DNA chip mounted on the working mounter in a desired sample, a power source part for applying a voltage to the electrode of the DNA chip so that the DNA makes reaction with the sample to cause electrochemiluminescence, and an optical detection part for detecting a light of the electrochemiluminescence to analyze the DNA.

The working mounter includes a central part having the DNA chip mounted thereon, the DNA with a plurality of probe DNAs different from one another mounted thereon, a reference electrode on one side of the DNA chip, and a counter electrode on the other side of the DNA chip.

The sample reservoir and the working mounter have the same shape.

Thus, method and device for detecting a DNA of the present invention has fast, and simple detection process and system, low device cost, and can detect accurately.

It is to be understood that both the foregoing general description and the following detailed description are exemplary and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this specification, illustrate embodiments of the invention and together with the description serve to explain the principles of the invention.

In the drawings:

FIGS. 1A-1D illustrate the steps of a method for detecting a DNA in accordance with a preferred embodiment of the present invention;

FIG. 2 illustrates a device for detecting a DNA in accordance with a first preferred embodiment of the present invention;

FIG. 3 illustrates an enlarged view of “A” part in FIG. 2;

FIG. 4 illustrates a device for detecting a DNA in accordance with a second preferred embodiment of the present invention;

FIG. 5 illustrates an enlarged view of “B” part in FIG. 4;

FIGS. 6A and 6B illustrate graphs showing oxidation-reduction reaction of intercalators and electrochemiluminescent reaction fluid of the present invention;

FIGS. 7A and 7B illustrate graphs showing electrochemiluminescence of intercalators and electrochemiluminescent reaction fluid of the present invention;

FIG. 8 illustrates a device for detecting a DNA in accordance with a third preferred embodiment of the present invention;

FIG. 9 illustrates an enlarged view of “C” part in FIG. 4; and,

FIG. 10 illustrates a device for detecting a DNA in accordance with a fourth preferred. embodiment of the present invention.

### DETAILED DESCRIPTION OF THE PREFERRED EMBODIMENT

Reference will now be made in detail to the preferred embodiments of the present invention, examples of which are illustrated in the accompanying drawings.

The present invention suggests a method in which a probe DNA (Deoxyribonucleic acid) is immobilized on a minute gold electrode by a method, such as self assembly, Langmuir Blodgett (LB), or the like, a complementary deoxyribonucleic acid (cDNA) is induced, to make hybridization with the probe DNA, an intercalator is bonded to a minor groove, major groove of a hybridized double-stranded DNA or non-covalently to hybridized double-stranded DNA, for detecting a reaction of a target DNA (cDNA) intended to detect from electrochemiluminescence reaction of a metal complex derivative of Tris(2.2′-bipridiyl)ruthenium(II)[Ru(bpy)₃²⁺] and the intercalator.

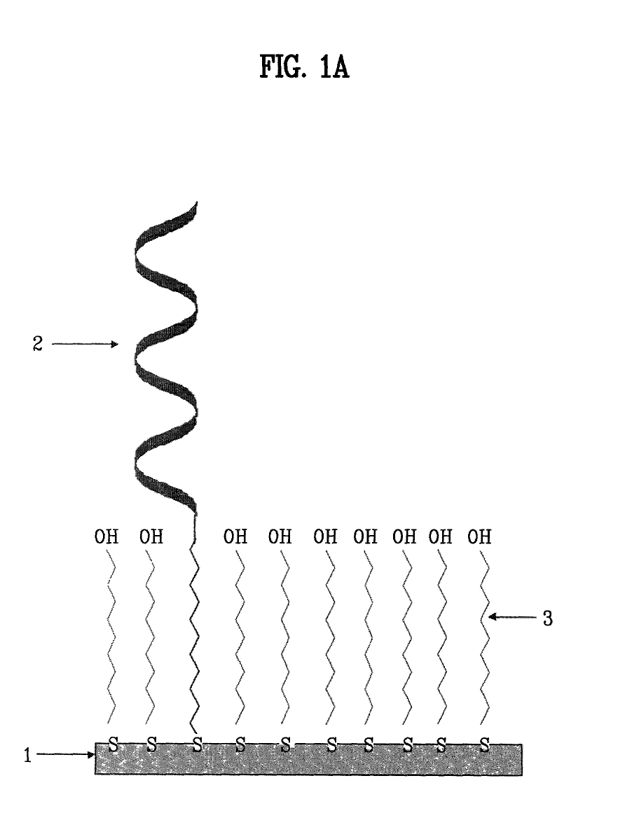

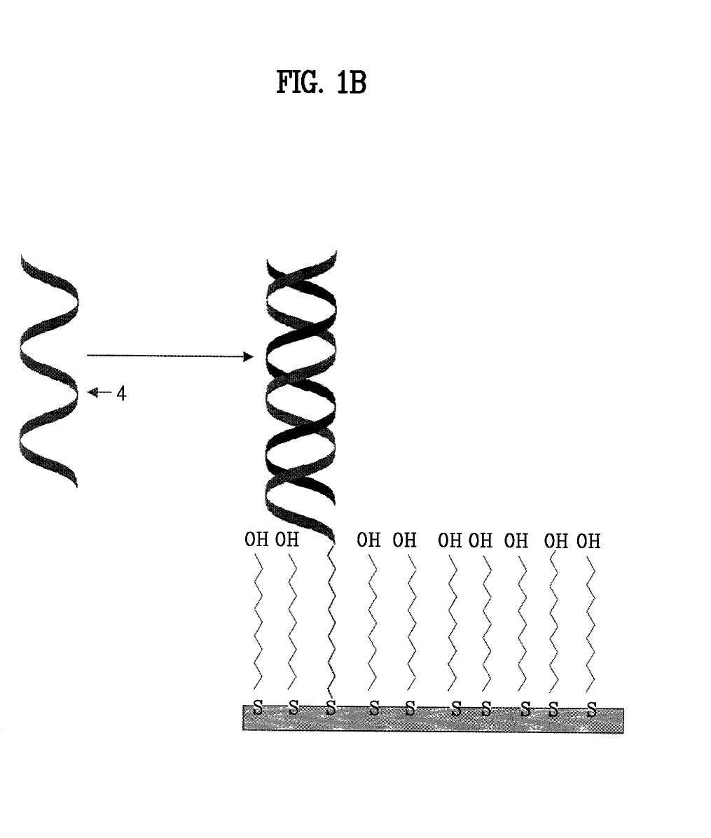

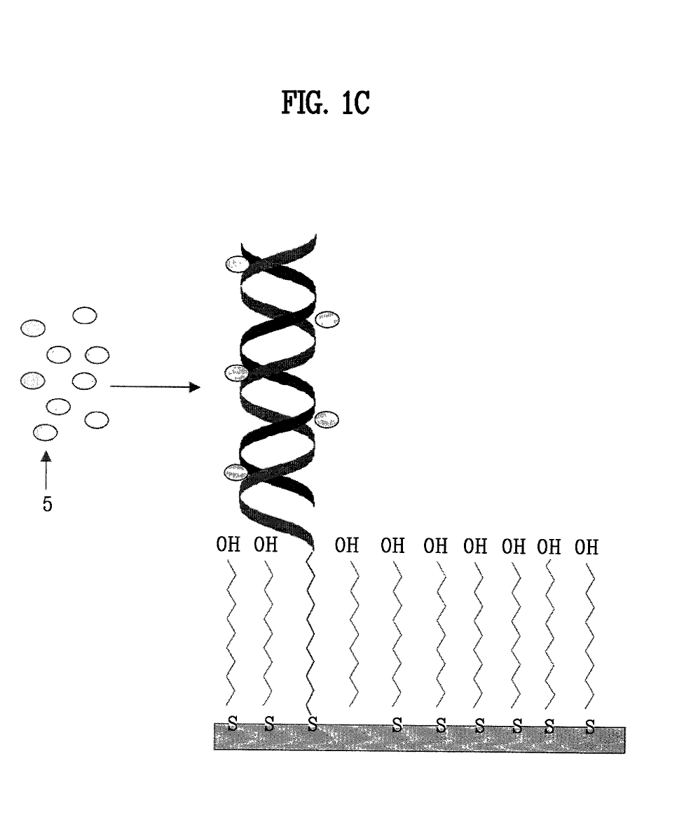

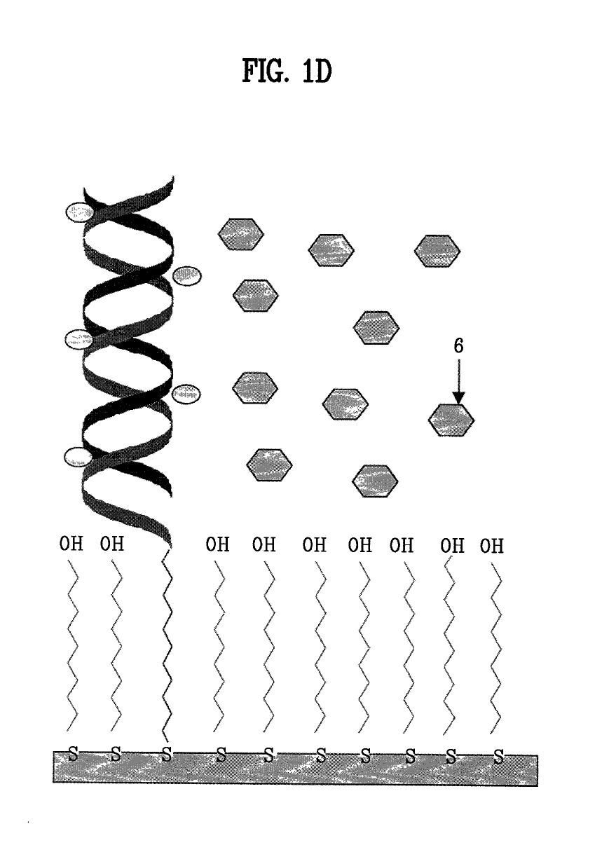

FIGS. 1A-1D illustrate the steps of a method for detecting a DNA in accordance with a preferred embodiment of the present invention.

Referring to FIG. 1A, a DNA chip is fabricated as follows.

A chrome layer (not shown) is coated on a substrate (not shown) of silicon to approx. 100 Å by vacuum deposition using an electron beam, and a gold electrode 1 is coated to approx. 1000 Å. The substrate may be formed of borosilicate glass.

Then, in order to immobilize a probe DNA 2 on a surface of the electrode 1, a washing process is conducted, in which the electrode is dipped in piranha solution for a few seconds for washing, and the electrode is washed with water, finally. The electrode 1 is dipped for approx. 10 min. in a mixed solution in which probe DNAs each having a thiol functional group at a 5′-phosphase position and ω-hydroxy-undecanethiol 3 are dissolved in an ethanol/octane mixed solvent at an 1:8 molar ratio, and the electrode 1 washed with ethanol, and water in succession, to fabricate a DNA chip having the probe DNA 2 immobilized on the surface of the electrode 1.

The ω-hydroxy-undecanethiol 3 prevents adsorption of non-specific DNA to the electrode 1 surface. However, because electron transfer reaction between the electrode and transition metal complex is difficult if an entire surface of the electrode 1 is covered with the self assembly material, either a time period of the self assembly reaction is regulated so that the self assembly material covers approx. an half of the electrode 1 surface, or 3-mercaptopropionic acid having a short hydrocarbon is employed instead of the ω-hydroxy-undecanethiol 3, for an easy electron transfer regardless of the electrode 1 surface coverage.

Next, referring to FIG. 1B, the target DNA 4 is introduced into the chip having the probe DNA 2 immobilized thereon, to hybridize the probe DNA 2 and the target DNA 4. In this instance, though the target DNAs 4 are hybridized with the probe DNAs 2 by complementary bonding, non-complementary target DNAs 4 make no hybridization with the probe DNAs 2. If a positive voltage is applied to the electrode 1, the target DNAs 4 charged with negative charge move faster toward the electrode, to make bond with the probe DNA 2, quickly. If a negative voltage is applied to the electrode 1 after the DNA hybridization ends, the non-complementary target DNAs 4 remained without being bonded with the probe DNAs 2 are removed with easy.

Then, referring to FIG. 1C, the intercalators 5 are fixed to the hybridized DNAs. In most of the cases, the intercalators 5 are bonded in minor grooves, major grooves, or between base pairs non-covalently. As a material for the intercalator 5, daunorubicin, nogalamycin, doxorubicin, and DAPI(4′,6-diamidino-2-phenylindole) or the like, are used.

Or, as the intercalator, a material may be used selected from a material obtained by bonding proline, oxalic acid, TPA (tripropylamine), or the like with Hoechst 33258, quinacrine, acridine orange, or the like. That is, a material that intercalates well is bonded with proline, oxalic acid, TPA, or the like that reacts with a material like Tris(2.2′-bipridiyl)ruthenium(II)[Ru(bpy)₃²⁺] well, a new intercalator 5 that intercalates well while making reaction with Tris(2.2′-bipridiyl)ruthenium(II)[Ru(bpy)₃²⁺] well can be prepared. After the intercalators 5 are thus fixed to the DNA, the intercalators are washed with a buffer solution, to remove intercalators 5 that are not fixed. In this instance, an amount of the intercalators 5 fixed to the DNA duplex is in proportion to an amount of DNAs hybridized on the electrode.

As a next step, referring to FIG. 1D, either Tris(2.2′-bipridiyl)ruthenium(II) [Ru(bpy)₃²⁺], or Tris(2.2′-bipridiyl)osmium(II)[Os(bpy)₃²⁺], an electrochemiluminescent reaction fluid 6, is introduced into the chip having the immobilized intercalators 5 thereon. Then, a preset voltage is applied to an electrode of the DNA chip, to make reaction between the intercalators 5 and the electrochemiluminescent reaction fluid 6, to induce electrochemiluminescence. The voltage applied is in a range of approx. 0.5-1.15V; if Tris(2.2′-bipridiyl)ruthenium(II) [Ru(bpy)₃²⁺] or Tris(1,10-phenanthroline) ruthenium(II) [Ru(phen)₃²⁺] is used as the electrochemiluminescent reaction fluid 6, approx. 1.15V is used, and if Tris(2.2′-bipridiyl)osmium(II) [Os(bpy)₃²⁺] is used as the electrochemiluminescent reaction fluid 6, approx. 0.6V is used. When such a voltage is applied, an oxidized Ru(bpy)₃³⁺ derivative, or Os(bpy)₃³⁺ derivative makes oxidation-reduction reaction with an intercalator intercalated in a doubled-stranded DNA, to produce an excited Ru(bpy)₃³⁺, or Os(bpy)₃³⁺ derivative, which emits a light of approx. 610 nm when the excited Ru(bpy)₃³⁺, or 460 nm when the excited Os(bpy)₃³⁺ derivative returns to a ground state. In this instance, the metal complex is involved in a cycle, in which the metal complex is returned to an original oxidized state of +2 value again, altered to an oxidized state of +3 value by the oxidation voltage applied to the electrode again, and emits a light as the metal complex makes reaction with the intercalator again.

Then, lastly, the emitted light is detected by means of optic detection device, to analyze the light.

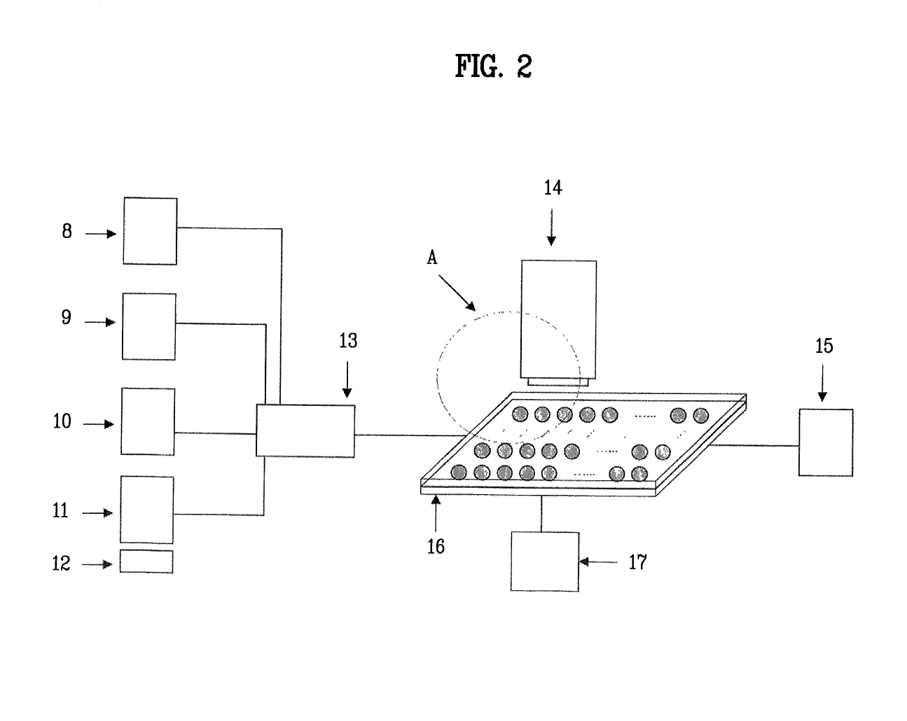

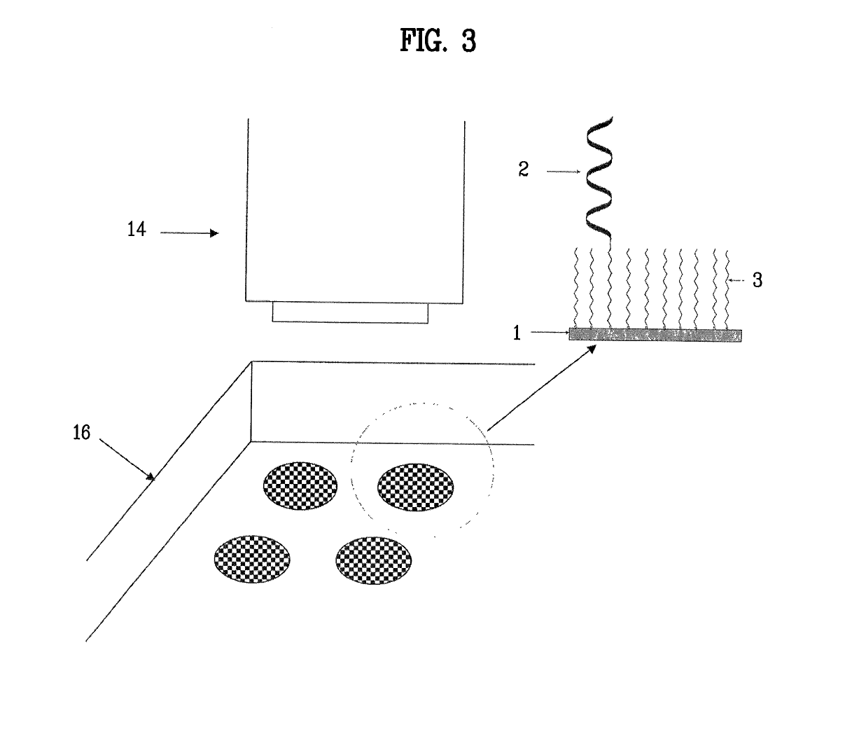

FIG. 2 illustrates a device for detecting a DNA in accordance with a first preferred embodiment of the present invention, and FIG. 3 illustrates an enlarged view of “A” part in FIG. 2.

Referring to FIG. 2, the device for detecting a DNA in accordance with a first preferred embodiment of the present invention includes a fastening part for fastening a DNA chip 16 having a plurality of probe DNAs different from one another on an electrode, a sample supplying part for supplying sample introduced for detecting a desired DNA from the DNA chip, an injecting part 13 for injecting the sample supplied from the sample supplying part to the DNA chip, a power source part 17 for applying a voltage to the electrode of the DNA so that the DNA makes reaction with the sample to cause electrochemiluminescence, an optical detection part 14 for detecting a light of the electrochemiluminescence, and a discarding part 15 for discarding unnecessary sample of the sample supplied to the DNA chip.

The sample supplying part includes an electrochemiluminescent reaction fluid supplying part 8 for supplying electrochemiluminescent reaction fluid, such as Tris(2.2′-bipridiyl)ruthenium(II) [Ru(bpy)₃²⁺], or Tris(2.2′-bipridiyl)osmium(II)[Os(bpy)₃²⁺], a buffer solution supplying part 9, an intercalator supplying part 10, and a target DNA supplying part 11.

For denaturing a DNA double strand, the target DNA may be heated at a heater 12 up to approx. 90-95° C., to denature the DNA double strand of the target DNA into single stranded DNAs. The injecting part 13 is a precision pump, or a dispenser, the power source part 17 is potentiostat, and as, the optical detection part 14, a photomultiplier tube (PMT), avalanche photodiode, cooled CCD camera, and the like, are used.

Referring to FIG. 3, the DNA chip 16 having a plurality of probe DNAs arranged on the electrode is fastened to a fixing part (not shown). The DNA chip 16 is used as a working electrode for applying a voltage, and, though not shown, Ag/AgCl electrode, or a silver wire is used as a reference electrode, and a platinum wire is built in as a counter electrode. The reference electrode, and the working electrode have the potentiostat in the power supplying part 17 connected thereto.

The operation of the foregoing device for detecting a DNA in accordance with a first preferred embodiment of the present invention will be explained.

Target DNAs are injected to the DNA chip 16 from the DNA supplying part through the injecting part 13, such as a precision pump, or a dispenser, and, after the target DNAs and the probe DNAs are hybridized adequately, intercalators are injected from the intercalator supplying part 10 to the DNA chip 16, for intercalation. Then, the intercalators not intercalated are removed by means of a buffer solution from the buffer solution supplying part 9, and Tris(2.2′-bipridiyl)ruthenium(II) [Ru(bpy)₃²⁺] is injected to the DNA chip 16, to induce electrochemiluminescence from Tris(2.2′-bipridiyl)ruthenium(II) [Ru(bpy)₃²⁺] and the intercalators. In a case Tris(2.2′-bipridiyl)ruthenium(II) [Ru(bpy)₃²⁺] is used as an electrochemiluminescent reaction fluid, approx. 1.15V is used, and in a case Tris(2.2′-bipridiyl)osmium(II) [Os(bpy)₃²⁺] is used as the electrochemiluminescent reaction fluid, approx. 0.6V is used. Then, a light of approx. 610 nm is emitted by the electrochemiluminescence, which is detected, and analyzed at the optical detection part 14, and remained sample on the chip 16 are discarded through the discarding part 15.

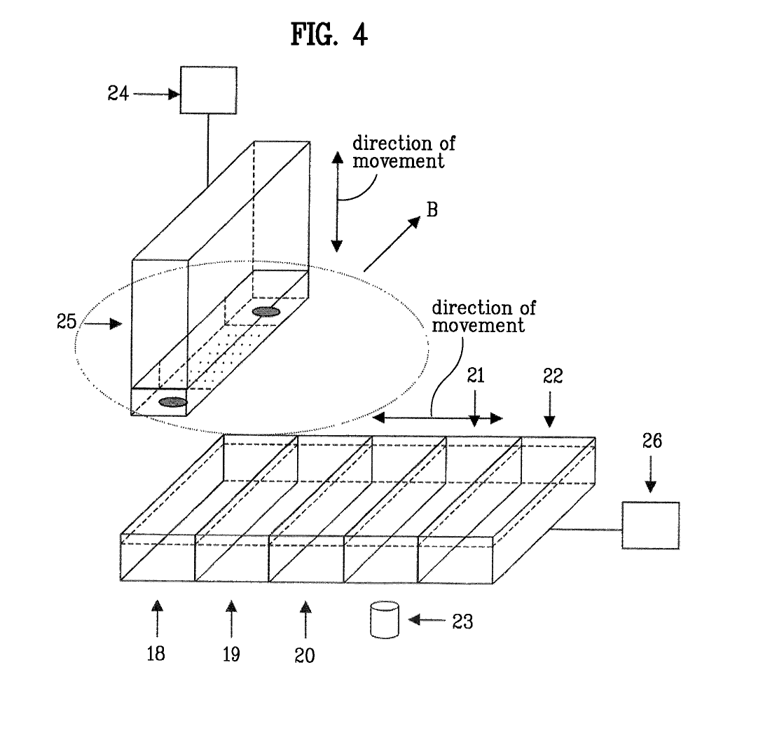

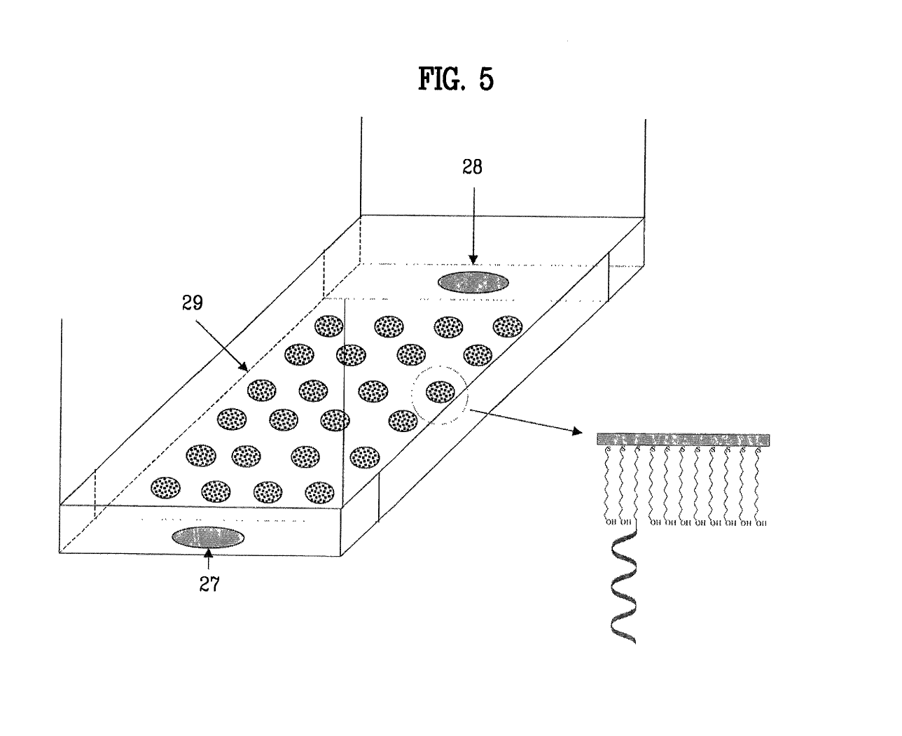

FIG. 4 illustrates a device for detecting a DNA in accordance with a second preferred embodiment of the present invention, and FIG. 5 illustrates an enlarged view of “B” part in FIG. 4. The device for detecting a DNA in accordance with a second preferred embodiment of the present invention is a device for automatic analysis of many DNAs while the DNA chip 29 is moved.

Referring to FIGS. 4 and 5, in the device for detecting a DNA in accordance with a second preferred embodiment of the present invention, a working mounter 25 is employed for fastening, and mounting a DNA chip having probe DNAs arranged thereon on an electrode, for making continuous analysis by moving up and down by an actuator 24, and automatic change of the DNA chip 29 of which analysis is finished upon finishing one running cycle process.

Referring to FIG. 5, the working mounter 25 has a central part for mounting a DNA chip 29 having a plurality of different probe DNAs arranged thereon, a reference electrode 27 on one side of the DNA chip 29, and a counter electrode 28 on the other side of the DNA chip 29.

In detection, at first, the working mounter 25 is moved to a target DNA reservoir 18 having target DNAs stored therein, for inducing complementary hybridization between the target DNA and the probe DNA. Then, the wording mounter 25 is moved to an intercalator reservoir 19 having intercalators therein, and intercalated at the intercalator reservoir 19. The reservoirs 18 and 19 are automatically moved by servo-motors, or step motors 26. Then, not intercalated intercalators are removed at a buffer solution reservoir 20, and electrochemiluminescence is induced from Tris(2.2′-bipridiyl)ruthenium(II)[Ru(bpy)₃²⁺] and the intercalator at a Tris(2.2′-bipridiyl)ruthenium(II) [Ru(bpy)₃²⁺] reservoir 21. A light from the electrochemiluminescence is detected, and analyzed at the optical detecting part 23, and the electrode passed through detection is washed at a washing reservoir 22, which is filled with 0.5M sulfuric acid solution.

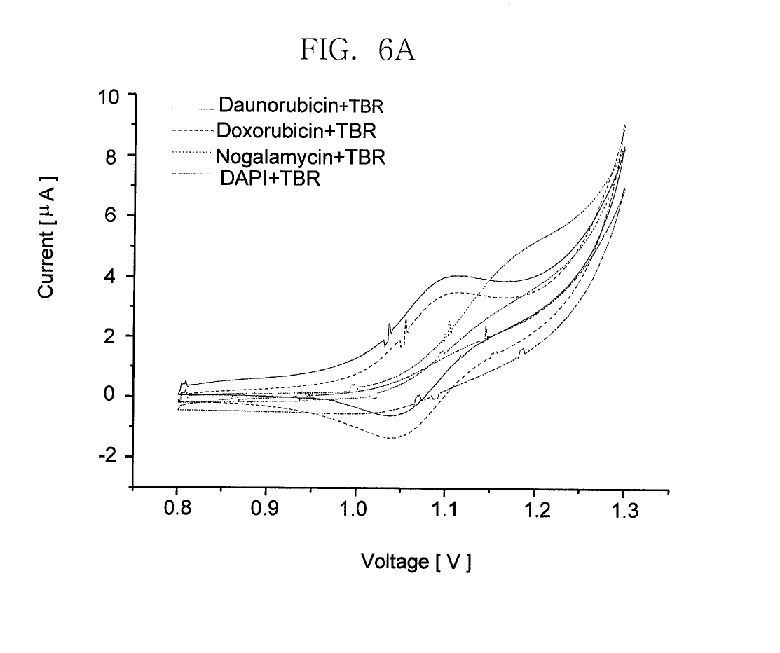

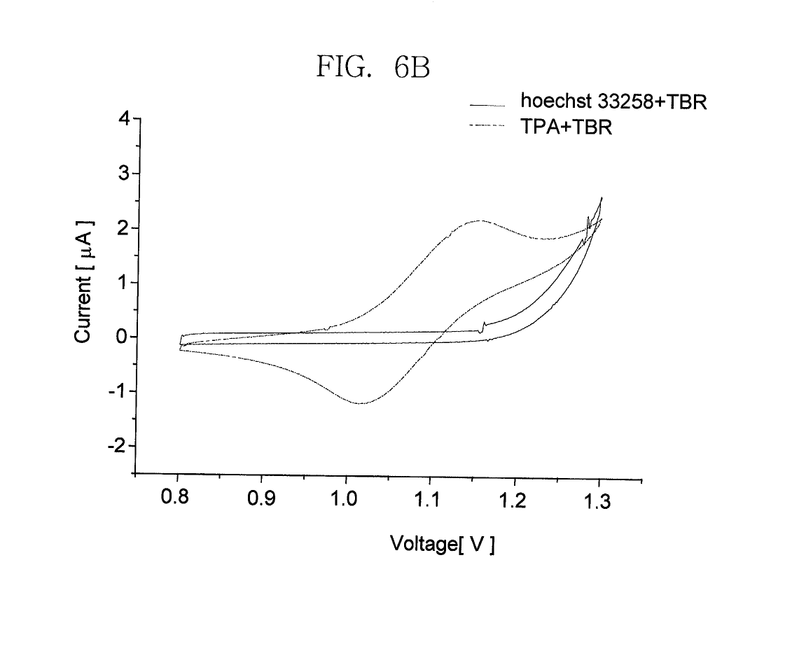

FIGS. 6A and 6B illustrate graphs showing oxidation-reduction reaction of an intercalator and electrochemiluminescent reaction fluid of the present invention. That is, FIG. 6A illustrates a result of conduction of cyclic voltammetry between daunorubicin, nogalamycin, doxorubicin, and DAPI(4′,6-diamidino-2-phenylindole), intercalators, and Tris(2.2′-bipridiyl)ruthenium(II)[Ru(bpy)₃²⁺] (TBR), an electrochemiluminescent reaction fluid. FIG. 6B illustrates a result of conduction of cyclic voltammetry between TPA(tripropylamine) and Tris(2.2′-bipridiyl)ruthenium(II)[Ru(bpy)₃²⁺] (TBR), an electrochemiluminescent reaction fluid or Hoechst 33258 and Tris(2.2′-bipridiyl)ruthenium(II)[Ru(bpy)₃₊] (TBR). Referring to FIG. 6A and FIG. 6B, it can be noted that daunorubicin, nogalamycin, doxorubicin, and DAPI(4′,6-diamidino-2-phenylindole) and Tris(2.2′-bipridiyl)ruthenium(II)[Ru(bpy)₃²⁺] (TBR), and TPA (tripropylamine) and Tris(2.2′-bipridiyl)ruthenium(II)[Ru(bpy)₃²⁺] have similar oxidation-reduction reaction. However, it can be noted that Hoechst 33258 and Tris(2.2′-bipridiyl)ruthenium(II)[Ru(bpy)₃²⁺] have no oxidation-reduction reaction.

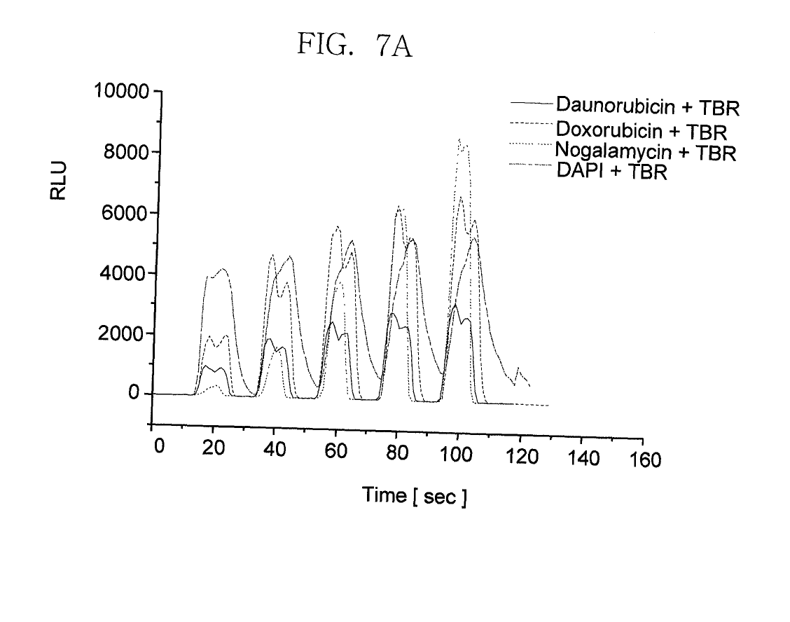

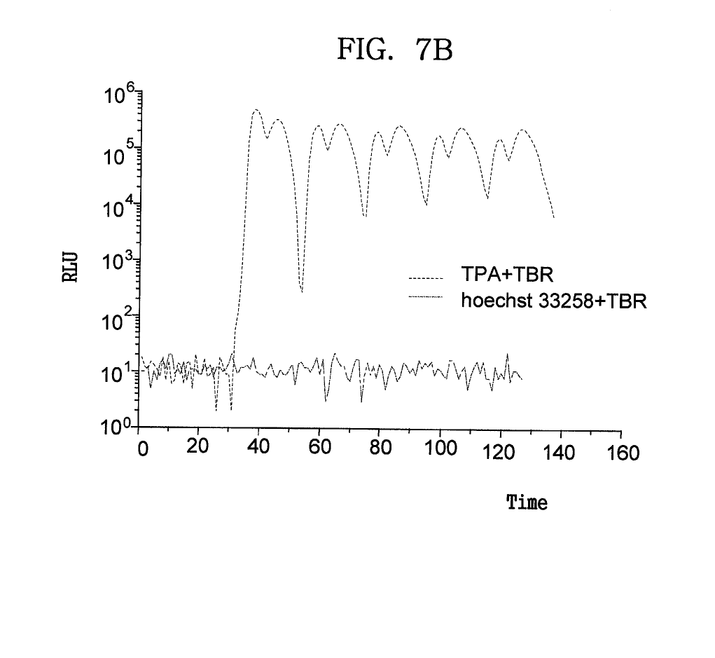

FIGS. 7A and 7B illustrate graphs showing electrochemiluminescence of intercalators and electrochemiluminescent reaction fluid of the present invention. That is, FIGS. 7A and 7B illustrate a result of detection by using a photomultiplier (PMT) while the cyclic voltammetry in FIGS. 6A and 6B is conducted.

Referring to FIGS. 7A and 7B, it can be noted that daunorubicin, nogalamycin, doxorubicin, and DAPI(4′,6-diamidino-2-phenylindole) and Tris(2.2′-bipridiyl)ruthenium(II)[Ru(bpy)₃²⁺] (TBR), or TPA (tripropylamine) and Tris(2.2′-bipridiyl)ruthenium(II)[Ru(bpy)₃²⁺] produce much light, Hoechst 33258 and Tris(2.2′-bipridiyl)ruthenium(II)[Ru(bpy)₃²⁺] produce no light. Thus, as the light is produced from electrochemiluminescence(ECL), the DNA can be detected by detecting the light.

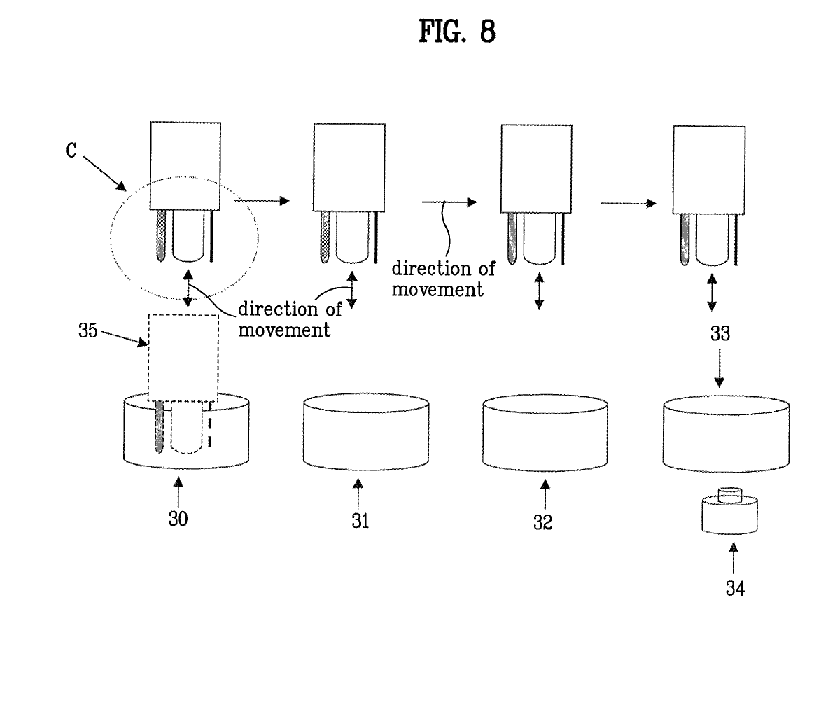

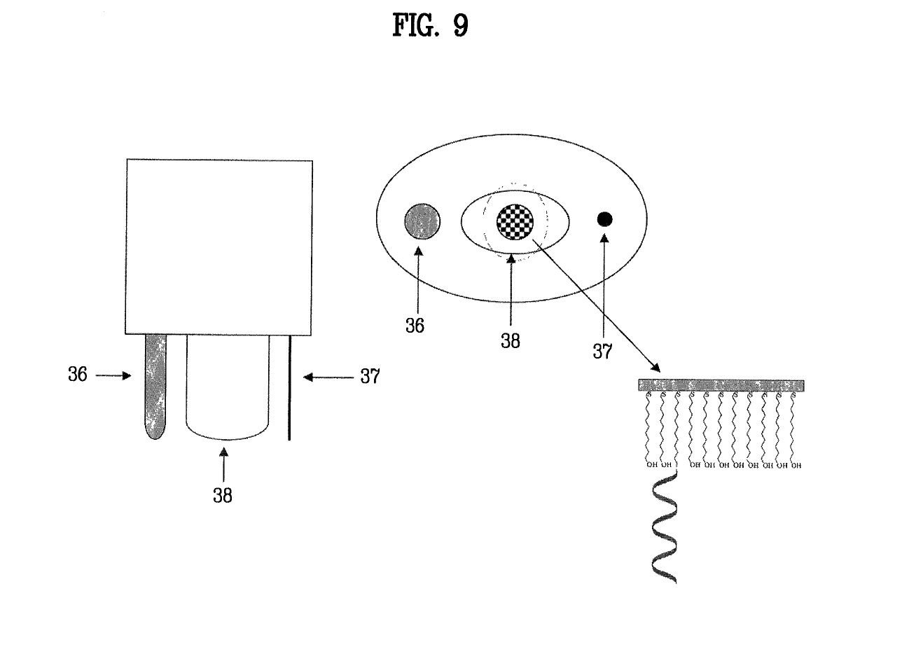

FIG. 8 illustrates a device for detecting a DNA in accordance with a third preferred embodiment of the present invention, and FIG. 9 illustrates an enlarged view of “C” part in FIG. 4, which is suitable for testing, or research as probe DNAs are arranged on one DNA chip.

Referring to FIG. 9, a working mounter 35 includes a central part having one DNA chip 38 fitted thereto, the DNA chip 38 having probe DNAs arranged thereon, a reference electrode 36 formed projected from one side of the DNA chip 38, and a counter electrode 37 formed projected from the other side of the DNA chip 38.

In detection, the working mounter 35 is moved to a target DNA reservoir 30 having target DNAs stored therein, for inducing complementary hybridization between the target DNAs and the probe DNAs. Then, the working mounter 35 is moved to an intercalator reservoir 31 having intercalators stored therein, for intercalation. Next, not intercalated intercalators are removed at a buffer solution reservoir 32, and electrochemiluminescence is induced from Tris(2.2′-bipridiyl)ruthenium(II)[Ru(bpy)₃²⁺] and the intercalator at a Tris(2.2′-bipridiyl)ruthenium(II) [Ru(bpy)₃²⁺] reservoir 32. A light from the electrochemiluminescence is detected, and analyzed at the optical detecting part 34.

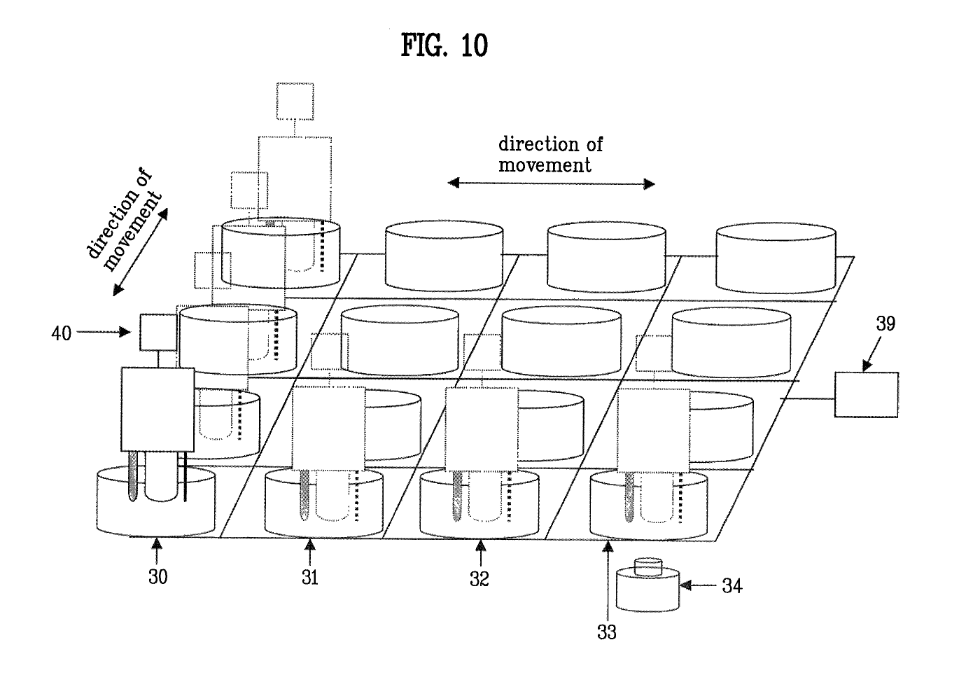

FIG. 10 illustrates a device for detecting a DNA in accordance with a fourth preferred embodiment of the present invention.

Referring to FIG. 10, the device for detecting a DNA in accordance with a fourth preferred embodiment of the present invention detects by a method identical to one in FIG. 9, except that the sample reservoirs are moved by a servo-motor, or a step motor 39.

As has been explained, the method and device for detecting a DNA of the present invention have the following advantages.

First, a DNA detection process is fast as no labeling reaction is required, which is required for the related art DNA chip or sensor, the DNA detection process is simple as no additional process is required.

Second, a DNA hybridization time period may be shortened by adjusting a voltage to an electrode.

Third, the use of electrochemiluminescence permits to dispense with an external light source, such as a laser, or a lamp, and a filter, a polarizer, or the like, that allows fabrication of a low cost DNA detection device.

Fourth, a precise detection of DNA is possible because there is no noise, or scattering of light caused by external light source, such as a laser, or the like.

Fifth, as the intercalators are intercalated at a DNA double strand formed by complementary reaction of DNAs, a precise, and selective DNA detection is possible.

Sixth, the automated processes, such as for the hybridization, and the like, make process convenient, and accurate. Once immobilization of many probe DNAs is made, many samples can be analyzed, quickly and conveniently.

It will be apparent to those skilled in the art that various modifications and variations can be made in the method and device for detecting DNA of the present invention without departing from the spirit or scope of the invention. Thus, it is intended that the present invention cover the modifications and variations of this invention provided they come within the scope of the appended claims and their equivalents.

What is claimed is:

1. A method for detecting DNA, comprising the steps of:
(a) immobilizing a probe DNA on a chip;
(b) placing a target DNA on the chip having the probe DNA immobilized thereon, for hybridization of the probe DNA and the target DNA;
(c) intercalating an intercalator to the hybridized DNA;
(d) introducing an electrochemiluminescent reaction fluid into the chip having the DNA with an intercalated intercalator;
(e) applying a preset voltage to the chip for causing reaction between the intercalator and the electrochemiluminescent reaction fluid; and,
(f) detecting, and analyzing a light from the reaction.

2. A method as claimed in claim 1, wherein the step (a) includes the steps of;
washing an electrode formed on the chip for the first time,
dipping the electrode in a mixed solution containing the probe DNA, and
washing the electrode for the second time for immobilizing the probe DNA on the electrode.

3. A method as claimed in claim 2, wherein the first time washing includes the steps of dipping of the electrode in piranha solution and water in succession, the mixed solution containing the probe DNA and ω-hydroxy-undecanethiol 3, or 3-mercaptopropionic acid are dissolved in an ethanol/octane mixed solvent, and the second time washing includes the steps of washing in ethanol and water.

4. A method as claimed in claim 2, wherein the electrode is formed of gold, the chip is formed of silicon, borosilicate glass, or PCB, and the probe DNA has a thiol functional group at a 5′-phosphate position.

5. A method as claimed in claim 1, wherein the intercalator is one selected from daunorubicin, nogalamycin, doxorubicin, and DAPI(4′,6-diamidino-2-phenylindole), or, one selected from a material obtained by bonding proline, oxalic acid, or TPA (tripropylamine) with Hoechst 33258, quinacrine, or acridine orange.

6. A method as claimed in claim 1, wherein the electrochemiluminescent reaction fluid is one selected from Tris(2.2′-bipridiyl)ruthenium(II)[Ru(bpy)₃²⁺], Tris(2.2′-bipridiyl)osmium(II)[Os(bpy)₃²⁺] and Tris(1,10-phenanthroline ruthenium(II) [Ru(phen)₃^{2+].}

7. A method as claimed in claim 1, wherein the step (b) includes the steps of;
placing the target DNA on the chip having the probe DNA immobilized thereon;
applying a first voltage to the chip for hybridizing the probe DNA and the target DNA; and
applying a second voltage to the chip for removing not hybridizing remained DNAs.

8. A method as claimed in claim 1, wherein the step (c) further includes the steps of
washing the intercalators not fixed to the hybridized DNA with a buffer solution.

9. A method as claimed in claim 1, wherein the preset voltage in the step (e) is 0.5-1.20V.

10. A device for detecting a DNA comprising:
a fastening part for fastening a DNA chip having a plurality of probe DNAs different from one another on an electrode;
a sample supplying part for supplying sample introduced for detecting a desired DNA from the DNA chip;
an injecting part for injecting the sample supplied from the sample supplying part to the DNA chip;
a power source part for applying a voltage to the electrode of the DNA so that the DNA makes reaction with the sample to cause electrochemiluminescence;
an optical detection part for detecting a light of the electrochemiluminescence to analyze the DNA; and
a discarding part for discarding unnecessary sample from the sample supplied to the DNA chip.

11. A device as claimed in claim 10, wherein the sample supplying part includes a electrochemiluminescent reaction fluid supplying part for supplying electrochemiluminescent reaction fluid, such as Tris(2.2′-bipridiyl)ruthenium(II) [Ru(bpy)₃²⁺], Tris(2.2′-bipridiyl)osmium(II)[Os(bpy)₃²⁺], a buffer solution supplying part, an intercalator supplying part, and a target DNA supplying part.

12. A device as claimed in claim 11, wherein the target DNA supplying part further includes a heater for heating the DNA double strand for denature the DNA double strand.

13. A device as claimed in claim 10, wherein the injecting part is a precision pump, or a dispenser, the power source part is potentiostat, the optical detection part is one selected from a photomultiplier tube (PMT), an avalanche photodiode, and a cooled CCD camera.

14. A device for detecting a DNA comprising:
a working mounter for fastening, and mounting a DNA chip having probe DNAs arranged thereon on an electrode;
a sample reservoir for storage sample introduced for detecting a desired DNA from the DNA chip;
a first driving part for moving the working mounter to the sample reservoir for dipping the DNA chip mounted on the working mounter in the sample;
a second driving part for moving the sample reservoir for dipping the DNA chip mounted on the working mounter in a desired sample;
a power source part for applying a voltage to the electrode of the DNA chip so that the DNA makes reaction with the sample to cause electrochemiluminescence; and,
an optical detection part for detecting a light of the electrochemiluminescence to analyze the DNA.

15. A device as claimed in claim 14, wherein the working mounter includes;
a central part having the DNA chip mounted thereon, the DNA with a plurality of probe DNAs different from one another mounted thereon,
a reference electrode on one side of the DNA chip, and
a counter electrode on the other side of the DNA chip.

16. A device as claimed in claim 14, wherein the working mounter includes;
a central part having one DNA chip mounted thereon, the DNA with a plurality of probe DNAs different from one another mounted thereon,
a reference electrode formed projected from one side of the DNA chip, and
a counter electrode formed projected from the other side of the DNA chip.

17. A device as claimed in claim 14, wherein the first driving part is an actuator, and the second driving part is a servo motor, or a step motor.

18. A device as claimed in claim 14, wherein the sample reservoir includes;
an electrochemiluminescent reaction fluid reservoir for supplying electrochemiluminescent reaction fluid, such as Tris(2.2′-bipridiyl)ruthenium(II) [Ru(bpy)₃²⁺], Tris(2.2′-bipridiyl)osmium(II)[Os(bpy)₃²⁺], and Tris(1,10-phenanthroline) ruthenium(II) [Ru(phen)₃²⁺], a buffer solution reservoir, an intercalator reservoir, a target DNA reservoir, and a washing fluid reservoir.

19. A device as claimed in claim 14, wherein the optical detection part is one selected from a photomultiplier tube (PMT), an avalanche photodiode, and a cooled CCD camera.

20. A device as claimed in claim 14, wherein the sample reservoir and the working mounter have the same shape.
